(19) United States
(12) Patent Application Publication
(10) Pub. No.: US 2024/0081375 A1
(43) Pub. Date: Mar. 14, 2024

(54) MICROBIAL CHINESE HERBAL MEDICINE FEED HAVING EFFECTS OF DETOXICATION,MUTTONY ODOR REMOVAL, FLAVOR ENHANCEMENT AND MUTTON QUALITY IMPROVEMENT

(71) Applicant: Xia ZHANG, Ordos, Inner Mongolia (CN)

(72) Inventors: Xia ZHANG, Ordos, Inner Mongolia (CN); Chuyue ZHANG, Ordos, Inner Mongolia (CN); Yao ZHANG, Ordos, Inner Mongolia (CN); Le KANG, Ordos, Inner Mongolia (CN)

(21) Appl. No.: 18/274,673

(22) PCT Filed: Jan. 14, 2022
(86) PCT No.: PCT/CN2022/071931

§ 371 Date: Jul. 27, 2023

(30) Foreign Application Priority Data
Dec. 16, 2021 (CN) 202111544466.4

(51) Int. Cl. A23K 10/30; A23K 50/10; A61K 36/185; A61K 36/21; A61K 36/232; A61K 36/235; A61K 36/315; A61K 36/42; A61K 36/484; A61K 36/52; A61K 36/53; A61K 36/539; A61K 36/575; A61K 36/61; A61K 36/67; A61K 36/734; A61K 36/752; A61K 36/758; A61K 36/8962; A61K 36/9064; A61K 36/9068; A61P 43/00

(52) U.S. Cl. CPC A23K 10/30; A23K 50/10; A61K 36/185; A61K 36/21; A61K 36/232; A61K 36/235; A61K 36/315; A61K 36/42; A61K 36/484; A61K 36/52; A61K 36/53; A61K 36/539; A61K 36/575; A61K 36/61; A61K 36/67; A61K 36/734; A61K 36/752; A61K 36/758; A61K 36/8962; A61K 36/9064; A61K 36/9068; A61P 43/00

(57) ABSTRACT

A microbial Chinese herbal medicine feed having effects of detoxication, muttony odor removal, flavor enhancement and mutton quality improvement, comprising 190-1920 parts of feed substrate, 39-238 parts of additives, 1-65 parts of Chinese herbal medicine components, and 100-550 parts of forage powder; the Chinese herbal medicine components comprise fructus aurantii, dried tangerine peel, Amomum villosum and so on. The formula of the invention can obviously decrease the synthesis of short-chain fatty acids and C18 fatty acids in sheep, inhibit and block the further combination of C6, C8 and C10 into stable complexes or associations to the greatest extent and effectively improve the quality of the mutton, particularly effective in removing odor of the mutton.

### TECHNICAL FIELD

The invention relates to the field of sheep feed, in particular to a series of microbial Chinese herbal medicine feeds which can realize the effects of “detoxification, mutton odor removal and aroma enhancement” of sheep through direct pharmacological action and secondary microbial deep reaction, and essentially improve the quality of mutton.

### BACKGROUND OF THE INVENTION

Mutton is one of the livestock meat widely eaten by consumers all over the world. Mutton is not only rich in nutrition, tender and easy to digest but also features high protein, low fat, more phospholipids and less cholesterol, at the same time, there are no religious and cultural taboos in the consumption of mutton. In recent years, Swiss scientists have found that a large amount of conjugated linoleic acid (CLA) in mutton has an obvious effect on preventing and curing cancer, whereas the special odor of mutton (especially during cooking) discourages many people; this unique flavor of mutton is called “Shan Wei” in China. It is precisely because the “mutton odor” is not generally accepted by consumers, which is the fundamental reason why mutton consumption can not be popularized all over the world.

The formation of “mutton odor” is influenced and controlled by many factors; scholars at home and abroad have divergent opinions on the formation of “mutton odor”, most foreign scholars hold the view that 4-methyloctanoic acid, 4-methylnonanoic acid and 4-ethyloctanoic acid are the main suppliers of “mutton odor”, whereas domestic academia maintains that the combination of hard fatty acids C18, C6, C8 and C10 in sheep under certain conditions to form stable complexes and associations is the leading cause of the formation of “mutton odor”.

Scholars at home and abroad have consistent views on the causes of the “special odor” of sheep: sheep variety, gender, age, feeding mode and forage selection can all affect the formation of “mutton odor”. The older the sheep, the greater the possibility of special odor formation; compared with captive sheep, the more likely the “odor of mutton” is to form; the above two items are superimposed.

Various institutions, scientific research institutes, enterprises and individuals at home and abroad have carried out a lot of researches and experiments on the removal of odor of mutton, it is classified into the following categories: 1. folk traditional mutton odor removal method, during cooking, the mutton is blanched or cooked with food that reduces the odor of mutton; 2. the method of removing the odor of mutton with Chinese herbal medicine, adding the corresponding Chinese herbal medicine to cook mutton; 3. physical and chemical methods to remove mutton odor, comprising high temperature heating method, rinsing mutton odor removal method, extrusion treatment and other deodorization methods; 4. microbial removal of mutton odor.

The common disadvantages of the prior arts: the above methods all carry out small-scale mutton odor removal treatment for the finished sheep ketone body after slaughter, they can not fundamentally remove the odor of mutton; moreover, it is impossible to realize large-scale industrialized production and promotion.

### SUMMARY OF THE INVENTION

The technical problem solved by the invention is to provide a synthetic feed of Chinese herbal medicine that improves the overall quality of mutton. Through the pharmacological action of the synthetic feed, the functions of the liver, spleen, stomach and intestines of the sheep are fundamentally improved and strengthened, and the intake of mutton is increased, it can also increase the in-depth decomposition and removal of harmful substances in ingested forage and toxins deposited in the body; enhance gastrointestinal motility, promote the digestion and absorption of nutrients and the excretion of harmful substances by sheep; and use Chinese herbal medicines to synthesize pharmacology Efficacy, improve the immunity of sheep, reduce the incidence of diseases, reduce the intake of chemical drugs and hormones due to treatment, and then obtain high-quality mutton of “detoxification, mutton odor removal, and aroma enhancement”.

To achieve the above objects, the invention adopts the following technical solutions:

A microbial Chinese herbal medicine feed for detoxifying, deodorizing, enhancing aroma and improving mutton quality the components are as follows in parts by weight:
190-1920 parts of feed substrate, 39-238 parts of additives, 1-65 parts of Chinese herbal medicine components and 100-550 parts of forage powder;
the components of Chinese herbal medicine comprise:
0.005-0.03 parts of Fructus aurantii, 0.003-0.021 parts of dried tangerine peel, 0.01-0.067 parts of Amomum villosum, 0.005-0.045 parts of Mongollian Thyme Herb, 0.003-0.053 parts of walnut, 0.007-0.035 parts of pepper, 0.015-0.075 parts of licorice, 0.005-0.02 parts of Angelica dahurica, 0.01-0.023 parts of round cardamom, 0.001-0.015 parts of Magnolia officinalis, 0.002-0.038 parts of Momordica grosvenori, 0.003-0.021 parts of Liquidambar, 0.002-0.017 parts of Chenopodium ambrosioides, 0.015-0.05 parts of hawthorn leaves, 0.003-0.018 parts of lysimachiae foenum-graeci herba, 0.01-0.025 parts of Scutellaria baicalensis, 0.015-0.065 parts of Radix isatidis, 0.018-0.059 parts of fig, 0.02-0.045 parts of hawthorn, 0.01-0.036 parts of Zanthoxylum bungeanum, 0.005-0.025 parts of dried ginger, 0.002-0.018 parts of seeds of Allium fistulosum, 0.01-0.031 parts of Fistular Onion Stalk, 0.001-0.02 parts of fennel, 0.005-0.19 parts of clove.

As an improvement, the feed substrate comprises one or more of corn, soybean, wheat bran, soybean meal, distiller's grains, peanut meal and sauce residue.

As an improvement, the proportions of the feed substrate are as follows in parts by weight:
50-670 parts of corn, 30-450 parts of soybean, 10-170 parts of wheat bran, 4-250 parts of soybean meal, 15-120 parts of distiller's grains, 30-150 parts of peanut meal, 50-110 parts of sauce residue.

As an improvement, the additives comprises one or more of bone meal, salt, pure plant charcoal powder and baking soda.

As an improvement, the proportions of the additives are as follows in parts by weight:
30-180 parts of bone meal, 3-25 parts of salt, 5-18 parts of pure plant charcoal powder, 1-15 parts of baking soda.

As an improvement, the forage powder is prepared from not less than three sorts of forage.

Fructus aurantii: slightly bitter, cold in nature, pungent and sour; it can help defecate, moisten intestines, expel gas and eliminate accumulated food from the body and has antibacterial and antioxidant effects.

Dried tangerine peel: it can encourage the secretion of digestive juice and eliminate the gas accumulated in the intestines and has the effects of enhancing aroma, strengthening the stomach and rest and recuperation.

Magnolia officinalis: bitter and pungent in taste and mild in nature, non-toxic; it mainly has the effects of strengthening the spleen, regulating the body, killing insects inside the stomach, generating muscles and facilitating blood circulation.

Amomum villosum: it mainly encourages the secretion of digestive juice, facilitates intestinal peristalsis, eliminates the accumulated gas in the intestine, and combines with tangerine peel, Magnolia officinalis and Fructus aurantii, which has the effect of regulating body, seasoning, brightening the eyes and invigorating the spleen. As a flavor material, can be used for both medicine and food, it has a strong aroma and can improve the meat flavor after being absorbed and precipitated in the body.

Mongollian Thyme Herb: pungent and warm in nature, slightly toxic; its volatile oil contains 53% Chenopodium ambrosioides, 4% zingiberene and thymol; main efficacy: it can dissipate chilliness, strengthen the spleen, help digest food, treat dyspepsia and improve the smell and flavor of the mutton.

Walnut: hot in nature, sweet in taste, mild in temperature, non-toxic; it can moisten blood vessels, make bones and flesh delicate, and deeply improve meat quality; in addition, ingesting walnut oil can increase protein concentration in the body and achieve rapid weight gain.

Licorice: sweet in taste and mild in nature; main efficacy: it can replenish spleen and regulate the body, clear away heat and detoxify; in addition, it can also play the role of antidote, ease the drug intensity and reconcile the drug properties in synthetic drugs.

Angelica dahurica: pungent, fragrant and bitter; it has the functions of facilitating blood circulation, expelling pus, generating muscle and relieving pain; its antibacterial effect plays an important role in the prevention and treatment of Escherichia coli and dysentery bacilli.

Round cardamom: pungent in taste, warm in nature and fragrant in odor; it can dry dampness, strengthen the spleen, warm the stomach and stop vomiting; in the invention, it is combined with other drugs to remove meat poison and peculiar odor.

Momordica grosvenori: cool in nature and sweet in taste; it has the effects of clearing heat and moistening lungs, smoothing intestines, defecating, clearing heat and detoxifying.

Liquidambar: mild in nature, pungent and slightly bitter in taste; it has the effects of facilitating blood circulation, relieving pain, detoxifying, generating muscle, cooling blood and removing toxins from the body.

Chenopodium ambrosioides: mild in nature, bitter and pungent in taste; it can expel wind and dehumidification, kill insects and relieve itching, facilitate blood circulation and reduce swelling.

Hawthorn leaves: mild in nature and sour in taste; it has the effects of facilitating blood circulation and removing blood stasis, regulating body and dredging pulse. In the invention, it mainly plays the role of removing turbidity and reducing fat.

lysimachiae foenum-graeci herba: mild in nature, pungent and sweet in taste; it has the functions of clearing heat, regulate the body, relieving pain and expelling insects.

Baikal Skullcap: cold in nature and bitter in taste; it can clearing away heat and dampness, clearing heat and detoxify, and has the effects of co-detoxifying and removing toxins combined with other drugs.

Radix isatidis: cold in nature and bitter in taste; it has the effects of clearing heat, detoxification, cooling blood and facilitating pharynx; combined with other drugs, it has the effect of co-detoxifying.

Fig: mild in nature and sweet in taste; it can strengthen the spleen, clear the intestines, reduce swelling, detoxify the body, boost immunity, and combined with other drugs to detoxify intestines and grass poison.

Hawthorn: mild in nature, sour and sweet in taste. It has the functions of helping digest food, strengthening spleen, regulating body and removing blood stasis. It is a kind of drug that helps digest food in the stomach. It can boost immunity and has antioxidative and antibacterial effects combined with other medicines.

Zanthoxylum bungeanum: warm in nature and pungent in taste. It has the functions of warming, relieving pain, killing insects and relieving itching. It also has the effects of sterilizing, inhibiting germ breeding and repelling insects combined with other medicines.

Dried ginger: hot in nature and pungent in taste. It has the functions of warming and dispersing chilliness, returning to Yang (one part of nature in Chinese culture) and dredging pulse, drying dampness and eliminating phlegm. It also has the effects of encouraging central excitement, strengthening stomach, antibacterial and aroma enhancement combined with other medicines.

Seeds of Allium fistulosum: warm in nature and pungent in taste. It can warm the kidney, brighten the eyes and detoxify; After combination, it can encourage the secretion of digestive enzymes, increase appetite, warm the stomach and facilitate the elimination of toxins in the body.

Fistular Onion Stalk: warm in nature and pungent in taste. It can invigorate Yang, detoxify, kill insects, resist bacteria, expel insects and increase aroma.

Fennel: mild in nature and pungent in taste. It can expel chilliness and relieve pain, regulate the body and stomach, and facilitate gastrointestinal peristalsis.

Cloves: pungent in taste and warm in nature. Efficacy and function: warming the stomach and the spleen, reducing stress, nurturing the kidney and helping us return to Yang; it also has the effects of combating stomach ulcer, relieving diarrhea and gallbladder, strengthening stomach, combating hypoxia and bacteria and killing insects.

The invention has the following advantages:

The invention adopts microbial Chinese herbal medicine feed, starting from living sheep breeding; according to the ruminant characteristics of sheep, after rumen digestion and ruminant process, the fully dissolved diversified elements are retained in the digestive system for a long time, under the action of a variety of digestive enzymes and through further microbial reaction of Chinese herbal medicine and the absorption and penetration of the digestive system of living sheep, the elements forming “mutton odor” in sheep are inhibited, so as to inhibit, reduce, decompose and remove the “mutton odor” from inside to outside; which can fundamentally improve the aroma of mutton and greatly improve the quality of mutton.

The formula of the invention can significantly reduce the synthesis of short chain fatty acids and C18 fatty acids in sheep, inhibit and block the further combination of C6, C8 and C10 into a stable complex or association compound to the greatest extent, can effectively improve the quality of mutton, and the “mutton odor removal” effect is particularly remarkable.

The sheep fed in the invention has bright wool, low incidence rate, high food intake and high rate of weight gain during a certain breeding period, after the sheep is slaughtered, the mutton is delicious, fragrant and has no odor of mutton, which can be more widely accepted by the majority of consumers.

### EMBODIMENTS OF THE INVENTION

The invention is described in detail and specifically through specific embodiments for you to better understand the invention, but the following embodiments do not limit the protection scope of the invention.

### Embodiment 1

This invention discloses a microbial Chinese herbal medicine feed for detoxifying, deodorizing, enhancing aroma and improving mutton quality, comprising:
feed substrate: corn 500 kg, soybean 400 kg, wheat bran 100 kg, soybean meal 150 kg;
additives: bone meal 90 kg, salt 20 kg, baking soda 7 kg;
forage powder: 250 kg;
components of Chinese herbal medicines: Fructus aurantii 8 g, dried tangerine peel 5 g, Amomum villosum 15 g, Mongollian Thyme Herb 17 g, walnut 1 g, pepper 31 g, licorice 21 g, Angelica dahurica 8 g, round cardamom 13 g, Magnolia officinalis 5 g, Momordica grosvenori 7 g, Liquidambar 8 g, Chenopodium ambrosioides 5 g, hawthorn leaves 19 g, lysimachiae foenum-graeci herba 11 g, Scutellaria baicalensis 13 g, Radix isatidis 23 g, fig, 19 g, hawthorn 23 g, Zanthoxylum bungeanum 14 g, dried ginger 7 g, seeds of Allium fistulosum 3 g, Fistular Onion Stalk 13 g, fennel 6 g, clove 7 g.

This embodiment is mainly used as feed for grazing free range sheep.

The production method of the embodiment is as follow: the raw materials are crushed respectively, and then the components of Chinese herbal medicine in the formula are crushed by an ultra-fine powder air mill and sieved through 150-200 mesh to obtain ultra-fine Chinese herbal medicine powder; Then, the feed matrix, additives, forage powder and Chinese herbal medicine were mixed evenly according to the proportion, and the appropriate dry wet ratio was achieved; The biological feed granules are formed by the feed expansion mechanism, and finally sealed with a moisture-proof bag.

### Embodiment 2

This invention discloses a microbial Chinese herbal medicine feed for detoxifying, deodorizing, enhancing aroma and improving mutton quality comprising:
feed substrate: corn 500 kg, soybean 350 kg, wheat bran 750 kg, distiller's grains 120 kg;
additives: salt 20 kg, baking soda 10 kg, pure plant charcoal powder 15 kg;
forage powder: 250 kg;
components of Chinese herbal medicines: Fructus aurantii 17 g, dried tangerine peel 11 g, Amomum villosum 25 g, Mongollian Thyme Herb 30 g, walnut 22 g, pepper 26 g, licorice 43 g, Angelica dahurica 15 g, round cardamom 25 g, Magnolia officinalis 1 g, Momordica grosvenori 13 g, Liquidambar 17 g, Chenopodium ambrosioides 9 g, hawthorn leaves 35 g, lysimachiae foenum-graeci herba 13 g, Scutellaria baicalensis 19 g, Radix isatidis 40 g, fig, 37 g, hawthorn 33 g, Zanthoxylum bungeanum 19 g, dried ginger 14 g, seeds of Allium fistulosum 11 g, Fistular Onion Stalk 20 g, fennel 12 g, clove 13 g.

This embodiment is mainly used as the feed for grazing free range sheep, and the preparation method is the same as that in embodiment 1.

### Embodiment 3

This invention discloses a microbial Chinese herbal medicine feed for detoxifying, deodorizing, enhancing aroma and improving mutton quality comprising:
feed substrate: corn 500 kg, soybean 250 kg, wheat bran 65 kg, soybean meal 100 kg, sauce residue 100 kg;
additives: salt 20 kg, baking soda 10 kg, pure plant charcoal powder 10 kg;
forage powder: 400 kg;
components of Chinese herbal medicines: Fructus aurantii 29 g, dried tangerine peel 18 g, Amomum villosum 54 g, Mongollian Thyme Herb 43 g, walnut 46 g, pepper 35 g, licorice 72 g, Angelica dahurica 19 g, round cardamom 43 g, Magnolia officinalis 15 g, Momordica grosvenori 19 g, Liquidambar 21 g, Chenopodium ambrosioides 15 g, hawthorn leaves 47 g, lysimachiae foenum-graeci herba 18 g, Scutellaria baicalensis 23 g, Radix isatidis 63 g, fig, 57 g, hawthorn 44 g, Zanthoxylum bungeanum 24 g, dried ginger 20 g, seeds of Allium fistulosum 7 g, Fistular Onion Stalk 31 g, fennel 17 g, clove 19 g.

This embodiment is mainly used as the feed for grazing free range sheep, and the preparation method is the same as that in embodiment 1.

### Embodiment 4

This invention discloses a microbial Chinese herbal medicine feed for detoxifying, deodorizing, enhancing aroma and improving mutton quality comprising:
feed substrate: corn 600 kg, soybean 250 kg, wheat bran 150 kg;
additives: bone meal 150 kg, salt 20 kg, baking soda 10 kg;
forage powder: 350 kg;
components of Chinese herbal medicines: Fructus aurantii 5 g, dried tangerine peel 4 g, Amomum villosum 17 g, Mongollian Thyme Herb 6 g, walnut 3 g, pepper 9 g, licorice 15 g, Angelica dahurica 6 g, round cardamom 11 g, Magnolia officinalis 3 g, Momordica grosvenori 4 g, Liquidambar 5 g, Chenopodium ambrosioides 7 g, hawthorn leaves 15 g, lysimachiae foenum-graeci herba 9 g, Scutellaria baicalensis 11 g, Radix isatidis 13 g, fig, 21 g, hawthorn 33 g, Zanthoxylum bungeanum 15 g, dried ginger 9 g, seeds of Allium fistulosum 9 g, Fistular Onion Stalk 1 g, fennel 9 g, clove 11 g.

This embodiment is mainly used as feed for captive sheep, and the preparation method is the same as that in embodiment 1.

### Embodiment 5

This invention discloses a microbial Chinese herbal medicine feed for detoxifying, deodorizing, enhancing aroma and improving mutton quality comprising:
feed substrate: corn 500 kg, soybean 250 kg, wheat bran 120 kg, distiller's grains 100 kg;
additives: salt 15 kg, pure plant charcoal powder 15 kg, baking soda 10 kg;
forage powder: 450 kg;
components of Chinese herbal medicines: Fructus aurantii 20 g, dried tangerine peel 17 g, Amomum villosum 34 g, Mongollian Thyme Herb 32 g, walnut 26 g, pepper 20 g, licorice 51 g, Angelica dahurica 13 g, round cardamom 28 g, Magnolia officinalis 9 g, Momordica grosvenori 15 g, Liquidambar 14 g, Chenopodium ambrosioides 13 g, hawthorn leaves 40 g, lysimachiae foenum-graeci herba 1 g, Scutellaria baicalensis 17 g, Radix isatidis 47 g, fig, 46 g, hawthorn 37 g, Zanthoxylum bungeanum 17 g, dried ginger 17 g, seeds of Allium fistulosum 13 g, Fistular Onion Stalk 18 g, fennel 15 g, clove 14 g.

This embodiment is mainly used as feed for captive sheep, and the preparation method is the same as that in embodiment 1.

### Embodiment 6

This invention discloses a microbial Chinese herbal medicine feed for detoxifying, deodorizing, enhancing aroma and improving mutton quality comprising:
feed substrate: corn 400 kg, soybean 250 kg, wheat bran 120 kg, sauce residue 90 kg;
additives: salt 15 kg, pure plant charcoal powder 15 kg, baking soda 10 kg;
forage powder: 550 kg;
components of Chinese herbal medicines: Fructus aurantii 27 g, dried tangerine peel 20 g, Amomum villosum 57 g, Mongollian Thyme Herb 39 g, walnut 50 g, pepper 31 g, licorice 69 g, Angelica dahurica 17 g, round cardamom 50 g, Magnolia officinalis 12 g, Momordica grosvenori 16 g, Liquidambar 20 g, Chenopodium ambrosioides 17 g, hawthorn leaves 50 g, lysimachiae foenum-graeci herba 15 g, Scutellaria baicalensis 25 g, Radix isatidis 59 g, fig, 60 g, hawthorn 40 g, Zanthoxylum bungeanum 30 g, dried ginger 24 g, seeds of Allium fistulosum 16 g, Fistular Onion Stalk 16 g, fennel 13 g, clove 17 g.

This embodiment is mainly used as feed for captive sheep, and the preparation method is the same as that in embodiment 1.

### Embodiment 7

This invention discloses a microbial Chinese herbal medicine feed for detoxifying, deodorizing, enhancing aroma and improving mutton quality comprising:
feed substrate: corn 600 kg, soybean 200 kg, wheat bran 100 kg, distiller's grains 70 kg, sauce residue 60 kg;
additives: bone meal 100 kg, salt 15 kg, pure plant charcoal powder 10 kg, baking soda 8 kg;
forage powder: 450 kg;
components of Chinese herbal medicines: Fructus aurantii 27 g, dried tangerine peel 20 g, Amomum villosum 57 g, Mongollian Thyme Herb 39 g, walnut 50 g, pepper 31 g, licorice 69 g, Angelica dahurica 17 g, round cardamom 50 g, Magnolia officinalis 12 g, Momordica grosvenori 16 g, Liquidambar 20 g, Chenopodium ambrosioides 17 g, hawthorn leaves 50 g, lysimachiae foenum-graeci herba 15 g, Scutellaria baicalensis 25 g, Radix isatidis 59 g, fig, 60 g, hawthorn 40 g, Zanthoxylum bungeanum 30 g, dried ginger 24 g, seeds of Allium fistulosum 16 g, Fistular Onion Stalk 25 g, fennel 13 g, clove 17 g.

This embodiment mainly for the mixed feeding of sheep and goats, and the production method is the same as that in embodiment 1.

The invention is provided with a control test:

1. Set Up the Experimental Group and the Control Group

Set up the experimental group:
(1) three goats and three lambs under the age of one were randomly selected as the feeding objects. Use the following feed with the formula of “detoxification, deodorization and aroma enhancement”:
among them, three goats and three lambs under the age of one were fed with the formula disclosed in embodiment 1 for three months. Three goats and lambs of one to three ages were fed with the formula disclosed in embodiment 2 for three months. Three goats and three lambs over the age of four were fed with the formula disclosed in embodiment 3 for three months.
(2) For captive sheep, under the reasonable breeding density, the minimum scale of 500 sheep is the experimental field, and this population is randomly selected;
Among them, three goats and three lambs under the age of one were fed with the formula disclosed in embodiment 4 for three months. Three goats and lambs of one to three ages were fed with the formula disclosed in embodiment 5 for three months. Three goats and three lambs over the age of four were fed with the formula disclosed in embodiment 6 for three months.
(3) From the selected free range group and captive group, three sheep and goats of three age groups were randomly selected, with a total of 36. They were fed on the same site with the formula feed disclosed in Example 7.

At the same time, the control group was set:
the method of selecting samples was exactly the same as that of the experimental group. The feed was a single corn straw kneading forage for three months.

2. Experimental Results

After the feeding, the experimental group and the control group were quarantined, tested and slaughtered, and then the mutton odor and peculiar odor of sheep were verified from six aspects: sheep fat, blood, viscera, sheep rumen sediment, sheep ketone body and deep mechanism sediment of sheep ketone body.

In this experiment, pure water was used as the medium for cooking for 60 minutes in a natural state without adding any seasoning. The personnel involved in the detection of mutton aroma were divided into three groups: professional experimental personnel, slaughtering professionals for more than three years and personnel without regular contact.

(1) Sheep Fat

The odor of fresh fat just slaughtered and frozen fat frozen for 30 days were evaluated.

After the isolation and verification of three groups of different personnel, it is summarized as follows:
sheep in the experimental group, fresh fat, frozen fat, dissolved fat; There is no odor of mutton and other peculiar odor in the three states.

Sheep of the same age in the control group in the same population were randomly selected: fresh fat, frozen fat and dissolved fat, all of which had mutton odor in three states; The odor of goat is stronger than that of sheep, the odor of fresh goat is greater after freezing, and the odor of older goat is stronger than that of younger goat.

(2) Blood

The odor of fresh blood and blood clots coagulated in boiling water within two minutes of slaughter sampling were evaluated.

After the isolation and verification of three groups of different personnel, it is summarized as follows:
in the experimental group, the fresh plasma had no odor of mutton, and had a slight odor of medicine; Boiling water coagulates and boils blood clots without odor of mutton.

Randomly selected sheep of the same age in the control group: fresh plasma has odor of mutton; There is a strong odor of mutton in the process of boiling blood clot.

(3) Internal Organs

The odor of visceral samples within three minutes after slaughter was evaluated.

After the isolation verification of three groups of different personnel, it is summarized as follows:

In the experimental group, the fresh viscera had bright color, clear vein, no odor of mutton and little peculiar odor.

Randomly selected sheep of the same age in the control group: fresh viscera have dark color, strong odor of mutton, fecal odor and other peculiar odor.

(4) Rumen Sediment

The rumen and its internal sediments within three minutes after slaughter were evaluated.

After the isolation and verification of three groups of different personnel, it is summarized as follows:
in the experimental group, there was no dark sediment between folds and bulges in the fresh rumen, with clear color and clear vein; the overall color is light yellow and green; Slightly sour, light sweet odor, no fecal odor.

Randomly selected sheep of the same age in the control group: there are a large number of dark sediments between folds and bulges in the fresh rumen; The overall color is dark green, with strong odor of mutton and heavy odor of feces.

(5) Sheep Ketone Body

Fresh sheep carcasses, sheep carcasses frozen for 30 days, frozen for 60 days and thawed were evaluated.

After the isolation verification of three groups of different personnel, it is summarized as follows:
in the experimental group, the fresh ketone body had clear color, clear vein, light drug odor and no odor of mutton; Mutton frozen for 30 days and 60 days has no odor of mutton in frozen and thawed state; The color of mutton frozen for 60 days is slightly darker.

In the same group, sheep of the same age in the control group were randomly selected: fresh ketone meat, frozen ketone meat and thawed ketone meat; Goat has stronger odor than sheep, fresh odor after freezing, older odor is stronger than younger odor, and ketone meat has the strongest odor after thawing.

(6) Detection of Deep Deposition of Sheep Ketone Body Texture

After the isolation verification of three groups of different personnel, it is summarized as follows:

In the experimental group, the fresh cut surface of ketone body muscle health has bright red color, clear texture, no odor of mutton, and has a strong mixed flavor of medicine and meat; After 30 days of freezing, there is no odor of mutton in the state of freezing, thawing and boiling; Under the natural thawing state at room temperature, the mixed aroma of medicine and meat is the strongest at the texture cutting section.

Randomly selected from the same group, the same age sheep in the control group: fresh ketone meat, frozen ketone meat, thawed ketone meat, after deep cutting of muscle health, there is a odor of mutton in all three parts, but the odor of mutton is slight compared with the parts with more fat deposition.

Finally, the contents of short chain fat and C18 fatty acids in the experimental group and the corresponding experimental group were tested by cold extraction technology and gas chromatography. The results are as follows:

TABLE 1
Contents of short chain fatty acids and C18 fatty acids in
muscle fat and subcutaneous fat of the experimental group

| | | | Fat in | | subcutaneous | |
|---|---|---|---|---|---|---|
| | | | muscle | | fat | |
| | | | SCFA | C18 | SCFA | C18 |
| goat | Sheep under | average value | 0.07 | 8.01 | 0.05 | 7.29 |
| | one age | standard deviation | 0.03 | 0.06 | 0.01 | 0.04 |
| | One to three | average value | 0.17 | 13.07 | 0.09 | 10.7 |
| | age sheep | standard deviation | 0.08 | 3.15 | 0.03 | 2.65 |
| | Sheep above | average value | 0.20 | 17.94 | 0.10 | 10.69 |
| | four age | standard deviation | 0.04 | 1.63 | 0.07 | 2.76 |
| sheep | Sheep under | average value | 0.09 | 9.37 | 0.06 | 9.15 |
| | one age | standard deviation | 0.04 | 0.08 | 0.02 | 0.06 |
| | One to three | average value | 0.20 | 16.31 | 0.12 | 13.17 |
| | age sheep | standard deviation | 0.09 | 4.53 | 0.05 | 4.97 |
| | Sheep above | average value | 0.23 | 19.43 | 0.23 | 14.03 |
| | four age | standard deviation | 0.07 | 2.79 | 0.11 | 5.21 |

TABLE 2
Contents of short chain fatty acids and C18 fatty acids
in muscle fat and subcutaneous fat in the control group

| | | | Fat in | | subcutaneous | |
|---|---|---|---|---|---|---|
| | | | muscle | | fat | |
| | | | SCFA | C18 | SCFA | C18 |
| goat | Sheep under | average value | 0.13 | 13.17 | 0.09 | 9.83 |
| | one age | standard deviation | 0.05 | 0.13 | 0.04 | 0.07 |
| | One to three | average value | 0.33 | 21.35 | 0.17 | 15.01 |
| | age sheep | standard deviation | 0.14 | 6.71 | 0.07 | 3.29 |
| | Sheep above | average value | 0.43 | 31.47 | 0.21 | 21.03 |
| | four age | standard deviation | 0.13 | 3.12 | 0.11 | 5.63 |
| sheep | Sheep under | average value | 0.17 | 19.01 | 0.08 | 17.58 |
| | one age | standard deviation | 0.09 | 0.17 | 0.05 | 0.13 |
| | One to three | average value | 0.41 | 33.14 | 0.27 | 21.38 |
| | age sheep | standard deviation | 0.17 | 9.02 | 0.08 | 9.07 |
| | Sheep above | average value | 0.47 | 37.56 | 0.43 | 26.73 |
| | four age | standard deviation | 0.15 | 5.43 | 0.26 | 11.05 |

It can be seen from the above table that, except for the small difference between the experimental group and the control group of goats under the age of one, the contents of short chain fatty acids and C18 fatty acids in the experimental group in other age groups and populations are far less than those in the control group, and the value is only close to half. Therefore, the above data can effectively prove that the formula of the invention can significantly reduce the synthesis of short chain fatty acids and C18 fatty acids in sheep, effectively improve the meat quality of sheep, and the “mutton odor removal” effect is particularly significant.

Meanwhile, during the feeding and observation process, researchers also found that the sheep in the invention has bright wool, low incidence rate, high food intake and high body weight gain in the unit breeding period, and the mutton after the slaughter was delicious, fragrant and tasty, which could be widely accepted by the consumers.

The specific embodiments of the invention have been described in detail above, but they are only used as examples, and the invention is not equivalent to the specific embodiments described above. For those skilled in the art, any equivalent modifications and substitutions of the invention are also within the scope of the invention. Therefore, the equal transformation and modification without departing from the spirit and scope of the invention should be included in the scope of the invention.

1. A microbial Chinese herbal medicine feed having effects of detoxication, muttony odor removal, flavor enhancement and mutton quality improvement, wherein the components are as follows in parts by weight:
190-1920 parts of feed substrate, 39-238 parts of additives, 1-65 parts of Chinese herbal medicine components and 100-550 parts of forage powder;
the components of Chinese herbal medicine comprise:
0.005-0.03 parts of Fructus aurantii, 0.003-0.021 parts of dried tangerine peel, 0.01-0.067 parts of Amomum villosum, 0.005-0.045 parts of Mongollian Thyme Herb, 0.003-0.053 parts of walnut, 0.007-0.035 parts of pepper, 0.015-0.075 parts of licorice, 0.005-0.02 parts of Angelica dahurica, 0.01-0.023 parts of round cardamom, 0.001-0.015 parts of Magnolia officinalis, 0.002-0.038 parts of Momordica grosvenori, 0.003-0.021 parts of Liquidambar, 0.002-0.017 parts of Chenopodium ambrosioides, 0.015-0.05 parts of hawthorn leaves, 0.003-0.018 parts of lysimachiae foenum-graeci herba, 0.01-0.025 parts of Scutellaria baicalensis, 0.015-0.065 parts of Radix isatidis, 0.018-0.059 parts of fig, 0.02-0.045 parts of hawthorn, 0.01-0.036 parts of Zanthoxylum bungeanum, 0.005-0.025 parts of dried ginger, 0.002-0.018 parts of seeds of Allium fistulosum, 0.01-0.031 parts of Fistular Onion Stalk, 0.001-0.02 parts of fennel, 0.005-0.19 parts of clove.

2. The microbial Chinese herbal medicine feed having the effects of detoxication, muttony odor removal, flavor enhancement and mutton quality improvement according to claim 1, wherein the feed substrate comprises one or more of corn, soybean, wheat bran, soybean meal, distiller's grains, peanut meal and sauce residue.

3. The microbial Chinese herbal medicine feed having the effects of detoxication, muttony odor removal, flavor enhancement and mutton quality improvement according to claim 2, wherein the proportions of the feed substrate are as follows in parts by weight:
50-670 parts of corn, 30-450 parts of soybean, 10-170 parts of wheat bran, 4-250 parts of soybean meal, 15-120 parts of distiller's grains, 30-150 parts of peanut meal, 50-110 parts of sauce residue.

4. The microbial Chinese herbal medicine feed having the effects of detoxication, muttony odor removal, flavor enhancement and mutton quality improvement according to claim 1, wherein the additives comprises one or more of bone meal, salt, pure plant charcoal powder and baking soda.

5. The microbial Chinese herbal medicine feed having the effects of detoxication, muttony odor removal, flavor enhancement and mutton quality improvement according to claim 4, wherein the proportions of the additives are as follows in parts by weight:
30-180 parts of bone meal, 3-25 parts of salt, 5-18 parts of pure plant charcoal powder, 1-15 parts of baking soda.

6. The microbial Chinese herbal medicine feed having the effects of detoxication, muttony odor removal, flavor enhancement and mutton quality improvement according to claim 1, wherein the forage powder is prepared from not less than three sorts of forage.
